(19) United States
(12) Patent Application Publication
(10) Pub. No.: US 2024/0034969 A1
(43) Pub. Date: Feb. 1, 2024

(54) MICROPLATE WELLS FOR CELL CULTIVATION

(71) Applicant: Molecular Devices (Austria) GmbH, Puch bei Hallein (AT)

(72) Inventors: Josef ATZLER, Hallein (AT); Andreas KENDA, Klagenfurt (AT); Felix SPIRA, Strasswalchen (AT)

(73) Assignee: Molecular Devices (Austria) GmbH, Puch bei Hallein (AT)

(21) Appl. No.: 18/258,721

(22) PCT Filed: Dec. 27, 2021
(86) PCT No.: PCT/IB2021/062356

§ 371 Date: Jun. 21, 2023

Related U.S. Application Data
(60) Provisional application No. 63/131,123, filed on Dec. 28, 2020.

(51) Int. Cl. C12M 1/32; C12M 1/04; C12M 1/00; C12M 3/00

(52) U.S. Cl. CPC C12M 23/12; C12M 23/24; C12M 23/22; C12M 23/46

(57) ABSTRACT

Disclosed are various embodiments for growing, culturing, monitoring, and analyzing embryoid bodies, fused embryoid bodies, spheroids, organoids, or other multi-cellular bodies in a microwell structure formed in one or more wells of an assay and culturing microplate. Hydrogel deposited into a well of the microplate and supported by a support ledge and the bottom surface of the well is molded into a microwell structure using a mold insert tool. In some examples, channels can be formed in the bottom of the microwell structure to allow for an exchange of fluid between a primary well section and a secondary well section of the well. The bottom surface of the assay and culturing microplate is optically transparent and gas-permeable.

### CROSS-REFERENCE TO RELATED APPLICATION

This application is being filed on Dec. 27, 2021 as a PCT International Patent Application and claims priority to and the benefit of U.S. Provisional Application Ser. No. 63/131,123, filed Dec. 28, 2020, entitled “MICROPLATE WELLS FOR CELL CULTIVATION,” the disclosure of which is hereby incorporated by reference herein in its entirety.

### BACKGROUND

Culturing cells in a three-dimensional (3D) environment yields cellular behavior and morphology that more closely matches what is observed in the human body. 3D hydrogels/hydroscaffolds used for this kind of culturing have a unique attribute: cells can be deposited in specific locations in 3D space and remain in position for extended time periods. This enables the creation of structures (e.g., embryoid bodies, fused embryoid bodies, spheroids, tumoroids, organoids, and/or other multi-cellular bodies) and co-culture environments where cellular interactions and developments over time are observed.

### SUMMARY

In one aspect, the technology relates to a microplate, including: a plate body having an array of wells; and a gas-permeable sheet secured to a lower portion of the array of wells, the gas-permeable sheet forming a bottom surface of at least a portion of each of the wells. In an example, the microplate further includes a clamping frame including an array of collars, individual ones of the collars being positioned around a lower portion of corresponding ones of the wells. In another example, the individual ones of the collars are coupled to corresponding ones of the wells via a friction fit. In yet another example, the individual wells of the array of wells include a primary well section and a secondary well section. In still another example, the primary well section and the secondary well section are fluidly connected to one another.

In another example of the above aspect, the gas-permeable sheet forms the bottom surface for the primary well section. In an example, individual wells of the array of wells include a support ledge protruding from an interior surface of at least one well wall. In another example, the support ledge is ring-shaped. In yet another example, the support ledge is offset from the bottom surface at a predefined distance. In still another example, the microplate further includes hydrogel disposed in the individual wells of the array of wells, the support ledge supporting the hydrogel within the individual wells.

In another example of the above aspect, the hydrogel is molded to include a plurality of microwells in the individual wells. In an example, the gas-permeable sheet is optically transparent.

In another aspect, the technology relates to a microplate, including: a plate body having an array of well units extending from a first end to a second end, individual ones of the well units being formed by at least one well wall and includes a support ledge that protrudes from an interior surface of the at least one well wall into a well opening, the support ledge being offset from the second end by predefined distance; and a gas-permeable sheet disposed on an underside of the individual ones of the well units at the second end thereby forming a bottom surface of the individual ones of the well units. In an example, the gas-permeable sheet is optically transparent. In another example, individual well units of the array of well units include a primary well section and a secondary well section. In yet another example, the primary well section and the secondary well section are fluidly connected. In still another example, the bottom surface of the primary well section includes the gas-permeable sheet.

In another example of the above aspect, the microplate further includes a clamping frame, the gas-permeable sheet being held against the underside of the individual ones of the well units via the clamping frame. In an example, the clamping frame further includes an array of collars, where individual ones of the collars are coupled to and positioned around a lower portion of a corresponding one of the well units. In another example, the individual ones of the collars are coupled to the corresponding ones of the well units via a friction fit. In yet another example, the support ledge is sized and positioned to provide support for an amount of hydrogel injected into the individual ones of the well units. In still another example, the support ledge is ring-shaped.

In another example of the above aspect, the microplate further includes hydrogel disposed in the individual well units of the array of well units, the support ledge supporting the hydrogel within the individual well units. In an example, the hydrogel is molded to include a plurality of microwells in the individual ones of the well units.

In another aspect, the technology relates to a kit, including: a microplate including an array of well units, individual ones of the array of well units includes a well body defined by at least one well wall that extends from a first end to a second end and an optically transparent viewing surface disposed at the second end, an interior of the at least one well wall includes a support ledge protruding from the at least one well wall into a well opening of a respective well unit, and the support ledge being offset from the second end by a predefined distance; and hydrogel for injecting into the individual well units of the array of well units, the support ledge being sized and shaped to support the hydrogel within the individual well units. In an example, the optically transparent viewing surface includes a gas permeable foil. In another example, the microplate further includes a clamping frame coupled to a lower portion of the array of well units, the gas permeable foil being positioned against the second end via the clamping frame. In yet another example, the individual ones of the well units include a primary well section and a secondary well section. In still another example, the primary well section is fluidly connected to the secondary well section.

In another example of the above aspect, the kit further includes a mold insert tool to form a plurality of microwells in the hydrogel. In an example, the mold insert tool includes a mold insert member being sized and shaped for insertion into a respective well unit of the array of well units. In another example, a shape of a cross-section of the mold insert member matches a shape of a cross-section of individual ones of the well units. In yet another example, a surface of a distal end of the mold insert member includes an arrangement of mold fingers. In still another example, the arrangement of mold fingers includes a square array of pyramids having an apex angle of about 32°.

In another example of the above aspect, the surface further includes a hollow extension disposed adjacent to the arrangement of mold fingers, the hollow extension being configured to form a pipetting channel. In an example, the mold insert member further includes a stop extension extending from an exterior surface of the mold insert member, the stop extension being configured to engage with the support ledge of the respective well unit upon insertion of the mold insert member into the respective well unit thereby restricting downward movement of the mold insert member into the well opening. In an example, the mold insert tool includes a plurality of mold insert members being in an arrangement that matches at least a portion of the array of well units. In another example, the hydrogel includes agarose. In yet another example, the hydrogel includes a first hydrogel, and further includes a second hydrogel. In still another example, the second hydrogel includes a poloxamer.

In another example of the above aspect, at a given temperature, the first hydrogel is in a gel form and the second hydrogel is in a liquid form. In an example, the given temperature is about 10 degrees Celsius (C) or less. In another example, at a given temperature, the second hydrogel is a gel, and the first hydrogel is a liquid. In yet another example, the first hydrogel transforms from a gel to a liquid as a temperature of the first hydrogel increases and the second hydrogel transforms from a gel to a liquid as a temperature of the second hydrogel decreases.

In another aspect, the technology relates to a method, including: depositing hydrogel into a microplate well, the microplate well includes a support ledge protruding from an interior surface of at least one well wall of the microplate well and being offset from a bottom surface of the microplate well by a predefined distance, and the hydrogel being supported by the support ledge and the bottom surface of the microplate well; and molding the hydrogel into a microwell structure includes a plurality of microwells. In an example, the method further includes inserting a mold tool into a well opening of the microplate well and causing the mold tool to engage with the hydrogel, the hydrogel being molded into the microwell structure according to a microwell mold configuration at a distal end of the mold tool. In another example, the hydrogel is in a gel form when the mold tool is inserted into the well opening, and further includes heating the mold tool to a temperature that causes portions of the hydrogel engaged with the mold tool begin to melt thereby causing the hydrogel to mold into the microwell structure. In yet another example, the hydrogel is in a liquid form when the mold tool engages with the hydrogel, and the hydrogel is cooled to a temperature that causes the hydrogel to gel prior to removal of the mold tool, the hydrogel being molded into the microwell configuration in response to the hydrogel being cooled while engaged with the mold tool. In still another example, the hydrogel includes a first hydrogel, and further includes injecting a second hydrogel into the microplate well prior to injecting the first hydrogel; and molding the second hydrogel into a channel configuration.

In another example of the above aspect, the first hydrogel is injected into the microplate well over the second hydrogel. In an example, the method further includes cooling the second hydrogel to cause the second hydrogel to transform into a liquid, the first hydrogel remaining a gel; and removing the second hydrogel thereby creating one or more channels within the gel of the first hydrogel, the one or more channels corresponding to the channel configuration of the second hydrogel. In another example, the second hydrogel is injected in a liquid form and molded into the channel configuration upon being transformed into a gel form In yet another example, the method further includes inserting a mold tool into a well opening of the microplate well and causing the mold tool to engage with the second hydrogel, the second hydrogel being molded according to a channel configuration at a distal end of the mold tool. In still another example, the first hydrogel includes agarose and the second hydrogel includes a poloxamer.

In another example of the above aspect, the microplate well includes an optically transparent gas-permeable bottom surface.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 illustrates an example of a perspective view of a microplate according to various embodiments of the present disclosure.

FIG. 2 illustrates an example of an exploded view of the microplate of FIG. 1 according to various embodiments of the present disclosure.

FIG. 3 illustrates an example of a bottom view of the microplate of FIG. 1 according to various embodiments of the present disclosure.

FIG. 4 illustrates an example of a cross-sectional view of the microplate of FIG. 1 according to various embodiments of the present disclosure.

FIG. 5 illustrates an example of a cross-sectional view of another embodiment of a microplate according to various embodiments of the present disclosure.

FIG. 6 illustrates an example of a mold insert tool according to various embodiments of the present disclosure.

FIG. 7 illustrates an example of a microwell structure formed in a well unit of the microplate of FIG. 1 using the mold insert tool of FIG. 6 according to various embodiments of the present disclosure.

FIGS. 8A-8C are example cross-sectional views of a well unit of the microplate of FIG. 1 and illustrate an example process for creating the microwell structure of FIG. 7 according to various embodiments of the present disclosure.

FIGS. 9A-9C are example cross-sectional views of a well unit of the microplate of FIG. 5 and illustrate an example process for creating the microwell structure of FIG. 7 according to various embodiments of the present disclosure.

FIGS. 10A-10G are example cross-sectional views of a well unit of the microplate of FIG. 1 and illustrate an example process for creating the microwell structure of FIG. 7 and channels within the microwell structure of FIG. 7 according to various embodiments of the present disclosure.

FIGS. 11A-11H illustrate an example process for creating the microwell structure of FIG. 7 within a well unit of the microplate of FIG. 5 according to various embodiments of the present disclosure. FIGS. 11A-11F and 11H illustrate example cross-sectional views of the microplate of FIG. 5 and FIG. 11G illustrates an example top view of the microplate of FIG. 5 with channels formed in a hydrogel used to form the microwell structure of FIG. 7 according to various embodiments of the present disclosure.

FIG. 12 illustrates a flowchart of an example method related to the creation of a microwell structure in a well unit of the microplate in accordance to various embodiments of the present disclosure.

FIG. 13 illustrates an example of an exploded perspective view of a microplate according to another embodiment of the present disclosure.

FIG. 14 illustrates an example of an exploded perspective view of a microplate according to another embodiment of the present disclosure.

### DETAILED DESCRIPTION

The present disclosure relates to growing, culturing, monitoring, and analyzing of embryoid bodies, fused embryoid bodies, spheroids, organoids, and/or other multi-cellular bodies in vitro using microwell well microplates. According to various embodiments of the present disclosure, hydrogel (e.g., agarose) deposited into individual wells of the microplate is formed into a microwell structure used to support the growth and maintenance of cell aggregates. In various examples, the microwell structure includes channels used to facilitate a gravitational exchange of media without disturbing the environment in the well and/or microwell structure of interest. In addition, individual ones of the microplate wells may comprise an optically-transparent bottom surface that also may be gas-permeable and that (1) serves as a viewing window for imaging the spheroids, organoids, or other cellular bodies being cultured in the microwell structure and (2) enables an increase of oxygen supply for the growing spheroids, organoids, or other cellular bodies in the microwell structure.

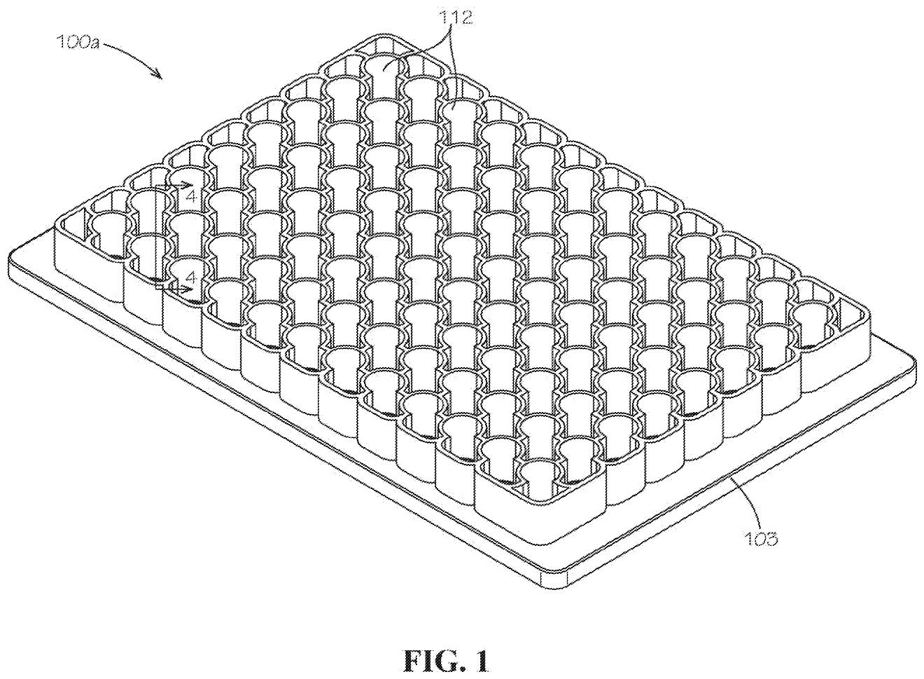

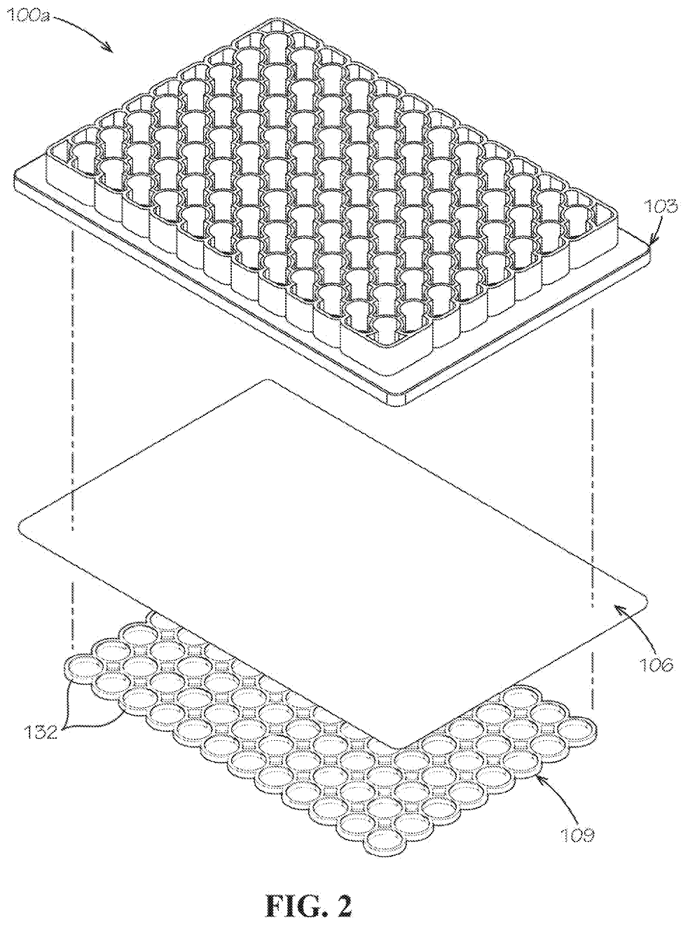

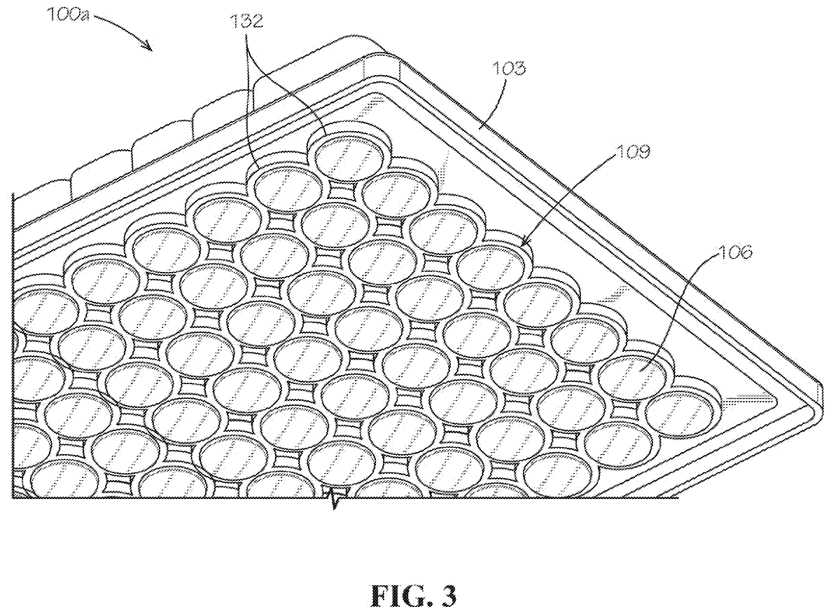

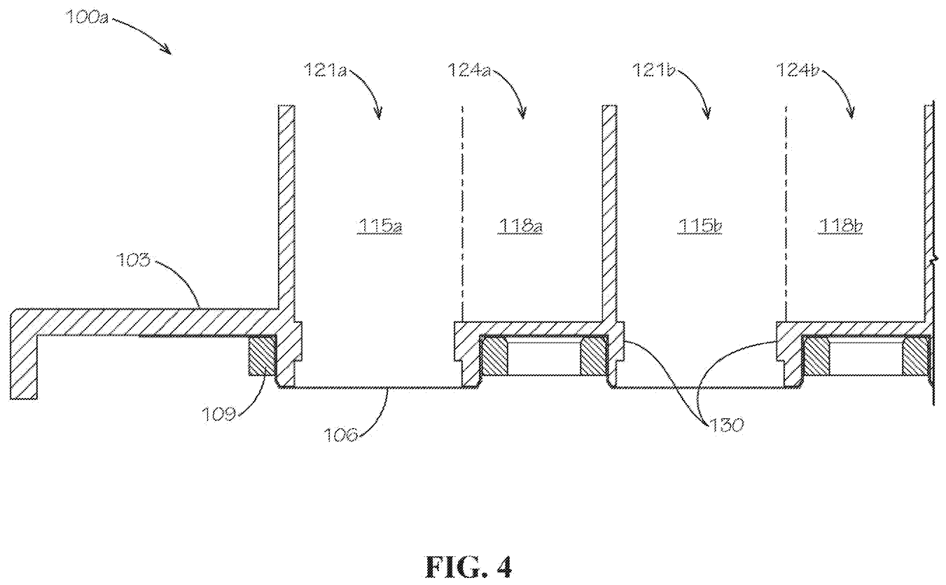

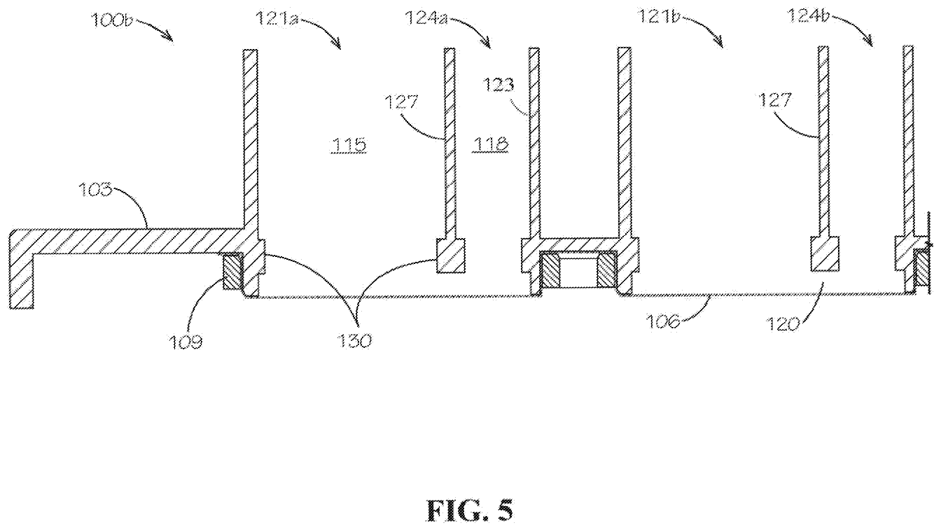

Turning now to FIGS. 1-5, shown are example views of a microplate 100 (e.g., 100a, 100b) that may be included in a kit, in accordance to various embodiments of the present disclosure. Other configurations of microplates are depicted elsewhere herein, but the various features and operations of growing and maintaining cell aggregates described further herein are described in conjunction with the microplate 100 of FIGS. 1-5, primarily for illustrative purposes. FIG. 1 illustrates an example perspective view of a microplate 100a. FIG. 2 illustrates an example of an exploded view of the microplate 100a including the well plate body 103, a bottom layer sheet 106, and a clamping frame 109, in accordance with various examples of the present disclosure. FIG. 3 illustrates a bottom view of the microplate 100a showing the bottom layer sheet 106 coupled to an underside of the well plate body 103 via the clamping frame 109. FIG. 4 illustrates a cross-sectional view of the microplate 100a of FIG. 1, in accordance with various examples of the present disclosure. FIG. 5 illustrates a cross-sectional view of another embodiment of the microplate 100b, in accordance with various embodiments of the present disclosure. As can be appreciated, the microplate 100 corresponds to a culturing and assay microplate for growing, culturing, monitoring, and assaying embryoid bodies, fused embryoid bodies, spheroids, organoids, or other multi-cellular bodies.

As shown in FIG. 1, the microplate 100a comprises a well plate body 103 having a plurality of well units 112 for growing, culturing, monitoring and assaying embryoid bodies, fused embryoid bodies, spheroids, organoids, and/or other multi-cellular bodies. In various examples, the well plate body 103 comprises a planar material having a top surface, a bottom surface, and a thickness corresponding to a desired well height. The components of the well plate body 103 may be formed of any suitable material by any suitable procedures. In exemplary embodiments, the well plate body 103 may be formed of polymer, such as a transparent polymer, and/or other material as can be appreciated. For example, the polymer may comprise polystyrene, polypropylene, poly(methyl methacrylate), cyclic olefin polymer, cyclic olefin copolymer, and/or other polymer as can be appreciated. In examples, acrylonitrile butadiene styrene (ABS) may be utilized. The well plate body 103 may have no removable/moving parts and/or may be formed as a single piece, such as by injection molding, such that all of the structures (e.g., wells) of the well plate body 103 are formed integrally with one another.

According to various embodiments, a well unit 112 comprises a primary well section 115 (e.g., 115a, 115b) (FIG. 4) and a secondary well section 118 (e.g., 118a, 118b) (FIG. 4). In various examples, the primary well section 115 and the secondary well section 118 can be fluidly connected with one another to facilitate a gravitational flow of liquid (e.g., feeding medium) between the primary well section 115 and the secondary well section 118 in response to a tilting of the microplate 100. For example, the primary well section 115 and the secondary well section 118 may be fluidly connected with one another via at least one channel 120 (FIG. 5) that is sized and shaped to facilitate the gravitational flow of liquid between the well sections. Exchanging the media between the primary well section 115 and the secondary well section 118 removes toxic by-products and supplies the growing cell cultures with fresh nutrients.

According to various embodiments, the primary well section 115 is sized and shaped to support deposited cell aggregates that may be embedded in hydrogel that is introduced into the primary well section 115. For example, the primary well section 115 may be considered a culture well that is used to grow the embryoid bodies, fused embryoid bodies, spheroids, organoids, and/or other multi-cellular bodies, as can be appreciated. According to various embodiments and dependent upon a number of well units 112 in the microplate 100, the width of the primary well section 115 can be up to about 8 millimeters (mm) (e.g., for 96 well plate), up to 11 mm (e.g., for a 48 well plate), up to about 17 mm (e.g., for a 24 well plate), and/or other sizes as can be appreciated. In addition, the depth of the primary well section 115 and the secondary well section 118 is specified such that the microplate 100 may be tilted to allow fluid exchange within the well units 112 without spilling the fluid out of the respective primary well section 115 or secondary well section 118 of each the well units 112.

The secondary well section 118 may be used to supply feeding media and/or other nutrients that can be used to feed the growing cell aggregates positioned in the primary well section 115. In addition, the secondary well section 118 can be used to harvest supernatant from the cell aggregates, as can be appreciated. For example, the secondary well section 118 can be considered a supply well that comprises the feeding media and/or other nutrients that may be used by the growing cell culture in the primary well section 115. The secondary well section 118 is sized and shaped to hold fluid that can be exchanged with the primary well section 115 according to various embodiments of the present disclosure. According to various embodiments and dependent upon a number of well units 112 in the microplate 100, the width of the secondary well section 118 can be up to about 8 millimeters (mm) (e.g., for 96 well plate), up to 11 mm (e.g., for a 48 well plate), up to about 17 mm (e.g., for a 24 well plate), and/or other sizes as can be appreciated.

According to various embodiments, the size and shape of the primary well section 115 and the secondary well section 118 may differ from one another. In some examples, the primary well section 115 is larger (in a dimension, for example diameter or volume) than the secondary well section 118. In other examples, the secondary well section 118 is larger than the primary well section 115. In some examples, the primary well section 115 comprises a shape that differs from a shape of the secondary well section 118.

The well units 112 are preferably arrayed in columns and rows as depicted in FIGS. 1-3. In various embodiments, the microplate 100 comprises a ninety-size (96) well-style plate comprising 96 primary well sections 115 for cell cultures as can be appreciated. However, it should be noted that the microplate 100 is not limited to a 96 well-style plate and can be organized as a strip, or other type of configuration as can be appreciated.

According to various embodiments, the primary well section 115 is defined by a primary well orifice 121 (e.g., 121a, 121b) (FIGS. 4 and 5) formed by one or more walls that extend from a top of the well plate body 103 to a bottom surface of the primary well section 115. Similarly, the secondary well section 118 is defined by a secondary well orifice 124 (e.g., 124a, 124b) (FIGS. 4 and 5) defined by one or more walls that extend from the top surface of the well plate body 103 to a bottom surface of the secondary well section 118. In various embodiments, the primary well section 115 is positioned adjacent to a secondary well section 118.

In some examples, as shown in FIG. 5, the primary well section 115 and the secondary well section 118 share a sidewall 127 or at least a portion of a wall shared between the primary well section 115 or the secondary well section 118. In various examples, the shared sidewall 127 of the primary well section 115 and the secondary well section 118 (or the portion of the wall shared between the primary well section 115 and the secondary well section 118) does not extend the entire length from the top surface to the bottom surface of the well plate body 103. In other examples, as illustrated in FIG. 4, the primary well section 115 and the secondary well section 118 do not share a wall and are partitioned by the primary well section 115 extending beyond a bottom surface of the secondary well section 118.

In further reference to FIG. 5, it should be noted that although the secondary well section 118 associated with the secondary well orifice 124b illustrates an opposing sidewall portion 123 from the shared sidewall 127 that is separated from the sidewall associated with the primary well section 115 of the adjacent well unit 112, in some embodiments, the opposing sidewall 123 is not present in the secondary well section 118 and/or is spaced at a distance from the adjacent well unit 112 that causes the secondary well orifice 124b to increase in volume.

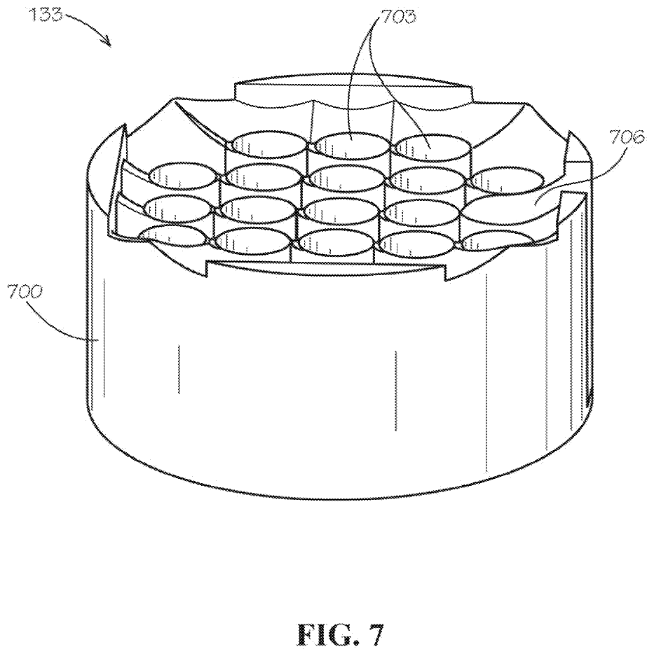

According to various embodiments, the primary well section 115 comprises a support ledge 130 (FIGS. 4 and 5) that protrudes into the primary well orifice 121 from an interior surface of at least one well wall defining the primary well section 115. According to various embodiments, the support ledge 130 is offset from a bottom surface of the primary well section 115 (e.g., the bottom layer sheet 106) by a predefined distance (e.g., within a range of 5 μm to 25 millimeters (mm)) such that the support ledge 130 is not flush with the bottom surface of the primary well section 115. In various embodiments, the support ledge 130 is sized and positioned within the interior surface of the primary well section 115 to provide support for hydrogel that is injected into the well and used to form a microwell structure 133 (FIG. 7) within the primary well section 115 of the corresponding well unit 112.

It should be noted that although the support ledge 130 is illustrated in the different embodiments of the microplate 100 in FIGS. 4 and 5, it should be noted that the support ledge 130 can be included in other microplates, as can be appreciated, including the microplates which are described in U.S. Provisional Application 63/094,946 entitled “Microplates for Automating Organoid Cultivation” filed on Oct. 22, 2020, which is incorporated by reference herein in its entirety.

According to various embodiments, the fluid connection between the primary well sections 115 and the adjacent secondary well sections 118 and the ability to provide a continual gravitational flow of fluid via the tilting of the microplate 100 allows for advance feeding of the cellular aggregates. In various examples, feeding media or other nutrients may be introduced into the secondary well section 118 and ultimately introduced into the primary well section 115 via the channel 120. In various embodiments, liquid can be removed from one of the well sections of the well unit 112 (e.g., secondary well section 118) by aspiration without disturbing the environment in the well of interest. In various examples, the fluid connection of the well sections of the well units 112 further allows for observation of the cell cultures in a hydrogel that may be in contact with two different liquids to create a gradient of concentrations within the hydrogel as can be appreciated.

According to various embodiments, the microplate 100 further comprises a bottom layer sheet 106 disposed on an underside of the well plate body 103. The bottom layer sheet 106 is attached to the underside of the well plate body 103 forming the bottom surface of the primary well sections 115. In some examples, as shown in FIG. 5, the bottom layer sheet forms the bottom surface of the primary well sections 115 and the secondary well sections 118. In other examples, the bottom surface of the secondary well sections 118 is formed via the well plate body 103 instead of the bottom layer sheet 106.

In various examples, the bottom layer sheet 106 comprises a viewing window that is optically transparent to allow for imaging of spheroids, organoids, or other cell cultures being cultured in the microplate 100, as can be appreciated. The viewing window can be a window that is suitable for microscopic observation, whether brightfield, phase-contrast, fluorescent, confocal, two-photon, or other microscopic imaging modalities as known in the art.

In various examples, the bottom layer sheet 106 comprises a gas permeable sheet that is configured to increase an oxygen supply for the growing spheroids, organoids, or other cellular bodies in the microplate 100. The gas permeable sheet can be formed of a material comprising polytetrafluoroethylene (PTFE), PEFP, polyimide, polydimethylsiloxane (PDMS), polypropylene (PP), polyvinyl chloride (PVC), cyclic olefin copolymer (COC) and/or other material as can be appreciated. According to various examples, the gas permeable sheet can have a thickness of about 5-30 microns or, in certain examples, about 25 microns. According to various examples, the gas permeable sheet may comprise a plurality of pores. In other examples, the gas permeable sheet may allow molecules to pass by diffusion. Alternatively, the gas permeable sheet may comprise some other thickness, pore diameter, and pore density.

According to various embodiments, the bottom layer sheet 106 is attached to the underside of the sidewalls of the primary well sections 115 and/or secondary well sections 118 of the well plate body 103 via the clamping frame 109. The clamping frame 109 comprises an array of collars 132 that are sized and shaped to engage with a lower portion of the well units 112 with the bottom layer sheet 106 disposed in between the clamping frame 109 and well plate body 103. In particular, the clamping frame 109 is designed to remain attached to and engaged with the well units 112 with the bottom layer sheet 106 disposed in between, thereby forming the bottom surface for the primary well sections 115 and/or secondary well sections 118. In various examples, the clamping frame 109 is attached to the well units 112 via a friction fit, thermocoupling, an adhesive, and/or other methods of attachment as can be appreciated.

In some examples, as shown in FIGS. 3 and 4, individual ones of the collars 132 of the clamping frame 109 are coupled to and positioned around a lower portion of the primary well section 115. In other examples, as shown in FIG. 5, the individual ones of the collars 132 of the clamping frame 109 are coupled to and positioned around a lower portion of a corresponding well unit 112 comprising both the primary well section 115 and the secondary well section 118.

It should be noted that although the bottom layer sheet 106 is discussed as being disposed along an underside of the well units 112 to form the bottom surface of the primary well section 115 and/or the secondary well section 118 via the clamping frame 109, in some embodiments, the clamping frame 109 is not required and the bottom layer sheet 106 is attached to the well plate body 103 via thermocoupling, an adhesive, and/or other methods of attachment as can be appreciated.

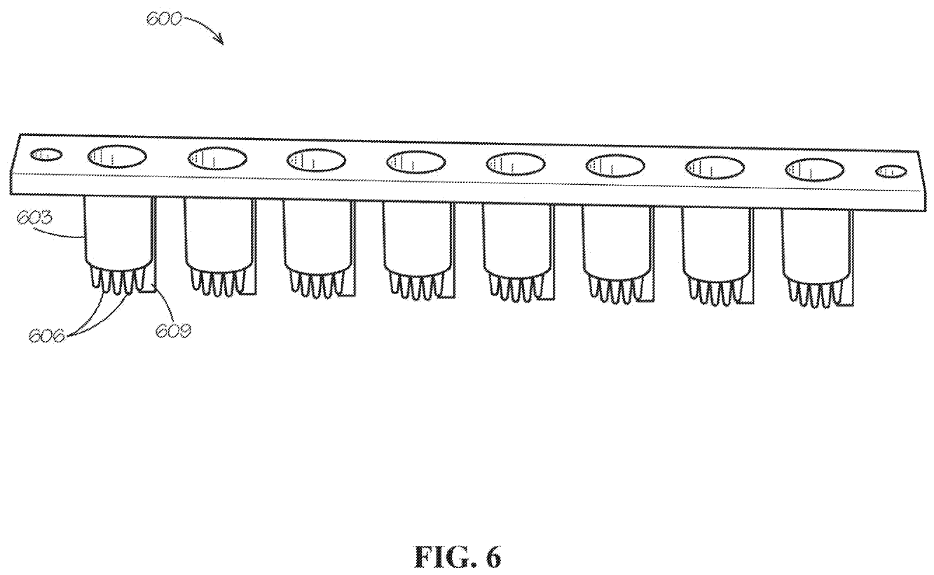

Turning now to FIG. 6, shown is an example of a mold insert tool 600 for molding hydrogel deposited within a well unit 112, in accordance with various embodiments of the present disclosure. In various examples, the mold insert tool 600 is designed to mold the hydrogel into the microwell structure 133 of FIG. 7. The mold insert tool 600 comprises one or more mold insert members 603 that are sized and shaped for insertion into a respective well unit 112. According to various examples, the arrangement of the one or more mold insert members 603 about the mold insert tool 600 can correspond to a single well unit 112, a row of well units 112, a column of well units 112, and/or an array of well units 112. The arrangement of the one or more mold insert members 603 allows for a simultaneous creation of microwell structures 133 in one or more well units 112 of a given microplate 100, as can be appreciated.

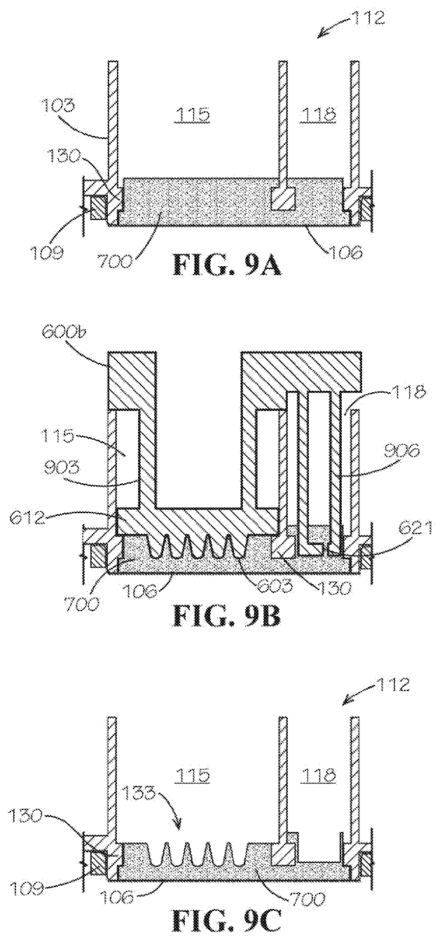

In various examples, a cross-section of the mold insert member 603 matches a shape of a cross-section of individuals ones of at least one of the primary well sections 115 and/or secondary well sections 118 of the well units 112. The example of FIG. 6, illustrates a mold insert member 603 with a cross-section matching a shape of a primary well section 115 of FIGS. 4 and 5. However, in various embodiments, the cross-section of the mold insert member 603 may correspond to a combination shape of the primary well section 115 and secondary well section 118 of the well units 112. In this example, a mold insert member 603 may comprise two extending insert members that correspond to the different sections of the well unit 112, as illustrated in FIGS. 9A-9C.

The mold insert member 603 of FIG. 6 comprises an arrangement of mold fingers 606 extending longitudinally from a distal end of the body of the mold insert member 603, according to various embodiments of the present disclosure. According to various embodiments, the mold fingers 606 are sized and shaped to form microwells in a hydrogel injected into a bottom of a well unit 112. In some examples, the arrangement of mold fingers 606 comprise a square array of pyramids. In some examples, the pyramids have an apex angle of about 32 degrees. However, the size, shape, and arrangement of the mold fingers 606 can vary based on the desired mold configuration.

In some examples, the mold insert member 603 further comprises a hollow extension 609 disposed adjacent to the arrangement of mold fingers 606. According to various examples, the hollow extension 609 is sized and shaped to form a pipetting channel in the microwell structure 133. It should be noted that although the mold insert member 603 provides an example of a mold configuration for the microwell structure 133 of FIG. 7, the mold insert member 603 may comprise other mold configurations for molding hydrogel in a desired configuration, as can be appreciated. For example, instead of an arrangement of mold fingers 606, the mold insert member 603 may comprise channels disposed within the distal end of the mold insert member 603. The channels may be used to form channels within a deposited hydrogel. In another example, the distal end of the mold insert member 603 may comprise a planar surface used to form a planar surface in the substance being molded.

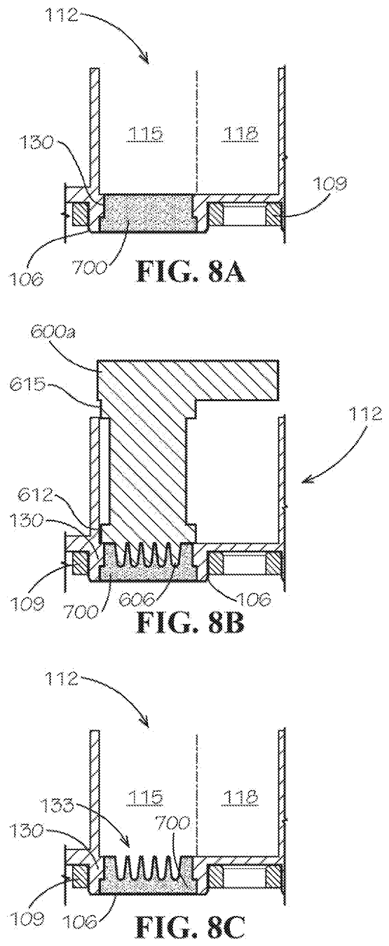

In various examples, the mold insert member 603 may comprise one or more lower stop extensions 612 (FIG. 8B) and/or one or more upper stop extensions 615 (FIG. 8B) extending radially from an exterior surface of the body of the mold insert member 603 and positioned at an offset from the distal end of the body of the mold insert member 603 by a respective predefined distance (e.g., within a range of 0 to 2 mm). According to various examples, the lower stop extension(s) 612 is configured to engage with the support ledge 130 upon insertion of the mold insert member into the respective well thereby restricting downward movement of the mold insert member 603 into the well opening. According to various examples, the upper stop extension(s) 615 is configured to engage with the top surface of the well plate body 103 surrounding the given well unit 112 upon insertion of the mold insert member 603 into the respective well unit 112 thereby restricting downward movement of the mold insert member 603 into the well opening. The lower stop extension 612 and the upper stop extension 615 are used to appropriately position the mold insert member 603 within the given well unit 112 for molding the hydrogel without damaging the hydrogel, as can be appreciated.

In some examples, the mold insert member 603 comprises a solid body. In other examples, the mold insert member 603 may comprise a hollow body (FIG. 9B). In examples where the mold insert member 603 comprises a hollow body, the mold insert member 603 may be used as a syringe for injecting hydrogel or other desired substance into a given well unit 112. For example, hydrogel may be inserted into the hollow region of the mold insert member 603 and injected into the bottom of the given well unit 112 via one or more apertures 621 (FIG. 9B) located at a distal end of body of the mold insert member 603. According to various examples, the mold insert member 603 may comprise a plunger (not shown) sized and shaped to telescopically fit within the hollow portion of the mold insert member 603. As the plunger is pushed (manually or automatically) towards the distal end of the mold insert member 603 and engages with the hydrogel, the hydrogel can be forced through the apertures 621 and into the well unit 112.

In various examples, the mold insert tool 600 is coupled to a temperature control device (not shown) that is configured to cool and/or heat the mold insert member(s) 603 to a given temperature. For example, in various embodiments, a mold insert member 603 may be inserted into a given well unit 112 having a liquid hydrogel deposited within. The mold insert member 603 may then be heated and/or cooled to the appropriate gelling temperature of the given hydrogel to allow the hydrogel to mold to the shape defined by the mold configuration of the mold insert member 603. In another example, the mold insert member 603 may be inserted into a given well unit 112 having a hydrogel that is in a gel formation. The mold insert member 603 may engage with the gelled hydrogel. The mold insert member 603 may then be heated or cooled to the liquifying temperature of the given hydrogel, thereby causing the areas of the hydrogel engaged with the mold insert member 603 to form into a shape defined by the mold configuration of the mold insert member 603. In other examples, the microplate 100 may be heated and/or cooled via a temperature control device in order to manipulate the gelling and/or liquification of the deposited hydrogel.

Moving on to FIG. 7, shown is an example of a perspective view of a microwell structure 133 formed using the mold insert tool 600 of FIG. 6, in accordance with various embodiments of the present disclosure. The microwell structure 133 comprises an arrangement of microwells 703 that are formed in a hydrogel 700 injected into a well unit 112 of a microplate 100. The microwell structure 133 further comprises a pipetting channel 706 for removing toxic by-products and supplying fresh nutrients to the growing cell culture without disrupting the environment in the well and/or microwell structure of interest. In various examples, the size of the pipetting channel 706 can be within a range of about 250 microns to 2 mm. In addition, the cross-section of the pipetting channel 706 can comprise a shape, as can be appreciated, including a circle, an ellipse, a square, a rectangle, and/or other shapes.

Turning now to FIGS. 8A-11H, shown are examples of how the microplate 100 and mold insert tool 600 may be used with regard to the formation of the microwell structure 133 as well as the growth and culturing of embryoid bodies, fused embryoid bodies, spheroids, organoids, and/or other multi-cellular bodies, in according to various examples of the present disclosure.

Starting with FIGS. 8A-8C, shown is an example of how hydrogel 700 deposited into a bottom of a primary well section 115 of a well unit 112 can be molded into a microwell structure 133, in accordance with various embodiments of the present disclosure. FIGS. 8A-8C illustrate a cross-sectional view of the well unit 112 of the microplate 100a, in accordance with various embodiments of the present disclosure. As shown in FIG. 8A, hydrogel 700 is deposited into a bottom of a primary well section 115 of the well unit 112. The deposited hydrogel 700 is supported by the bottom layer sheet 106 and the support ledge 130 of the primary well section 115. The hydrogel 700 can be deposited into the well using any suitable technique. In various examples, the hydrogel 700 comprises agarose, Polyethylenglycol (PEG), and/or other suitable substances, as can be appreciated.

In FIG. 8B, shown is an example of a mold insert member 603 of a mold insert tool 600a being inserted into the primary well orifice 121 of the primary well section 115 of a well unit 112, in accordance with various examples of the present disclosure. In the example of FIG. 8B, the mold fingers 606 engage with the hydrogel 700 that is situated at the bottom of the primary well section 115 and is supported by the support ledge 130 and bottom layer sheet 106. In addition, the lower stop extension 612 is engaged with the upper surface of the support ledge 130 and the upper stop extension 615 is engaged with the top surface of the well plate body 103 thereby restricting downward movement of the mold insert member 603 further into the well opening and controlling the amount of the deposited hydrogel 700 being molded.

In some examples, the deposited hydrogel 700 is in a liquid form and as the hydrogel 700 cools to the gelling temperature of the hydrogel 700, the portions of the hydrogel 700 engaged with the mold insert member 603, form into a configuration defined by the mold configuration of the mold insert member 603. In other examples, the injected hydrogel 700 is a gel configuration. In this example, the mold insert member 603 can be heated up to a melting temperature of the hydrogel (e.g., greater than about 88° Celsius (C) for agarose) causing the portions of the hydrogel 700 engaged with the mold insert member 603 to melt, thereby forming the microwell structure 133.

FIG. 8C illustrates an example cross-section of a well unit 112 of the microplate 100a of FIG. 1 following the removal of the mold insert tool 600 in accordance with various embodiments of the present disclosure. In particular, FIG. 8C illustrates the formation of the microwell structure 133 for growing cell cultures, as can be appreciated.

Turning now to FIGS. 9A-9C, shown is an example of how hydrogel 700 injected into a bottom of a primary well section 115 of a well unit 112 of the microplate 100b can be molded into a microwell structure 133, in accordance with various embodiments of the present disclosure. FIGS. 9A-9C differ from 8A-8C in that FIGS. 9A-9C illustrate a cross-sectional view of the well unit 112 of the microplate 100b and illustrate another embodiment of the mold insert tool 600b, in accordance with various examples of the present disclosure. As shown in FIG. 9A, hydrogel 700 is deposited into a bottom of a primary well section 115 of the well unit 112. The deposited hydrogel 700 is supported by the bottom layer sheet 106 and the support ledge 130 of the primary well section 115. The hydrogel 700 can be deposited into the well unit 112 using any suitable technique.

In FIG. 9B, shown is an example of a mold insert member 603 of a mold insert tool 600b being inserted into a well unit 112 of the microplate 100b, in accordance with various examples of the present disclosure. In the example of FIG. 9B, the mold insert member 603 comprises a first insert extension 903 and a second insert extension 906 corresponding to the primary well section 115 and secondary well section 118, respectively. The cross-section of the first insert extension 903 matches a shape of the primary well section 115 while the cross-section of the second insert extension 906 matches a shape of the secondary well section 118. In this example, the distal end of the first insert extension 903 comprises the mold fingers 606 engaged with the hydrogel 700 situated in the primary well section 115 and supported by the support ledge 130 and bottom layer sheet 106. Similarly, the distal end of the second insert extension 906 may comprise a difference configuration and is engaged with the hydrogel 700 situated in the secondary well section 118. Although the cross-section of the second insert extension 906 of FIG. 9B illustrates a planar configuration, the configuration can comprise any shaped configuration as desired to mold the hydrogel 700 in the secondary well section 118. For example, the second insert extension 906 may comprise a channel configuration to form channels within the hydrogel 700 in the secondary well section 118 to allow for a fluid connection between the primary well section 115 and the secondary well section 118, as can be appreciated.

FIG. 9B further illustrates the lower stop extension 612 being engaged with the upper surface of the support ledge 130 and the upper stop extension 615 being engaged with the top surface of the well plate body 103 thereby restricting downward movement of the mold insert member 603 further into the well opening and controlling the amount of the deposited hydrogel that will be molded.

It should be noted that the mold insert tool of FIG. 9B illustrates a mold insert tool 600b where the first insert extension 903 and the second insert extension 906 comprise a hollow body. As previously discussed, the hydrogel 700 may be injected into the well unit 112 via the hollow bodies of the first insert extension 903 and the second insert extension 906 of the mold insert tool 600b, as can be appreciated.

In some examples, the hydrogel 700 deposited into the bottom of the well unit 112 is in a liquid form. As the hydrogel 700 cools to the gelling temperature of the hydrogel 700, the portions of the hydrogel 700 engaged with the mold insert member 603b form into a configuration defined by the mold configuration of the mold insert member 603. In other examples, the injected hydrogel 700 is in a gel form. In this example, the mold insert member 603 can be heated up to a melting temperature of the hydrogel 700 causing the portions of the hydrogel engaged with the mold insert member 603 to melt, thereby forming the hydrogel in the microwell configuration of the mold insert member 603.

FIG. 9C illustrates an example cross-section of a well unit 112 of the microplate 100b of FIG. 1 following the removal of the mold insert tool 600b in accordance to various embodiments of the present disclosure. In particular, FIG. 9C illustrates the formation of the microwell structure 133 for growing cell cultures in the primary well section 115, as can be appreciated.

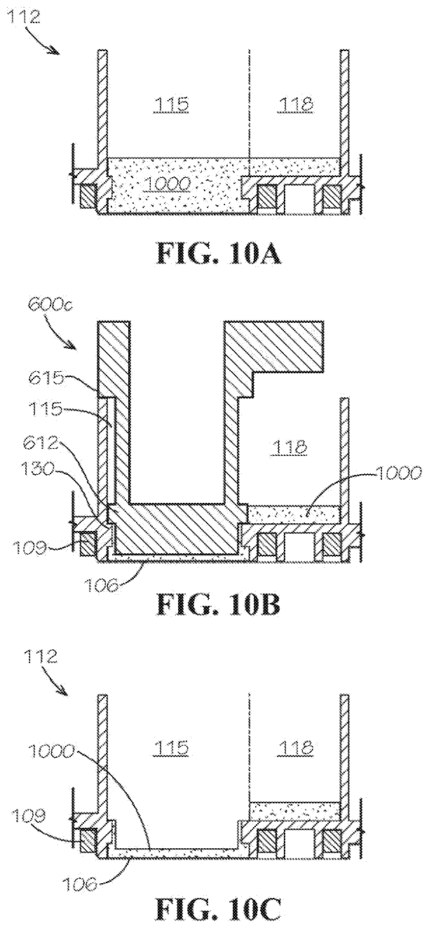

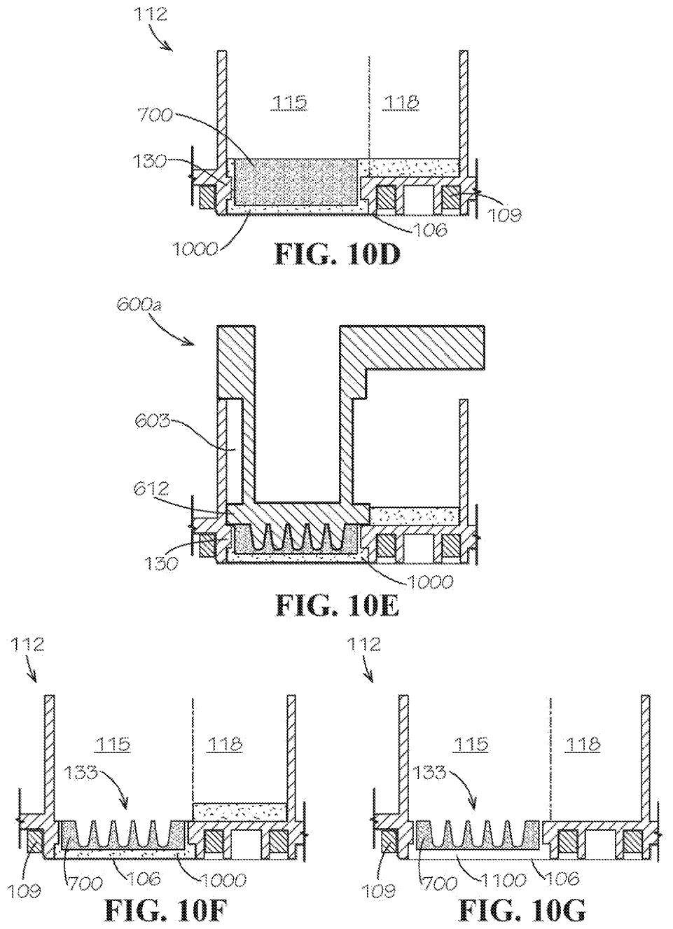

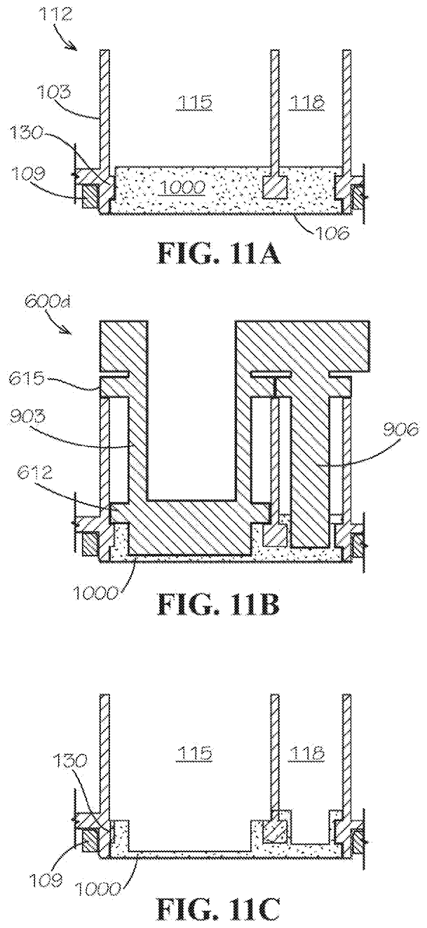

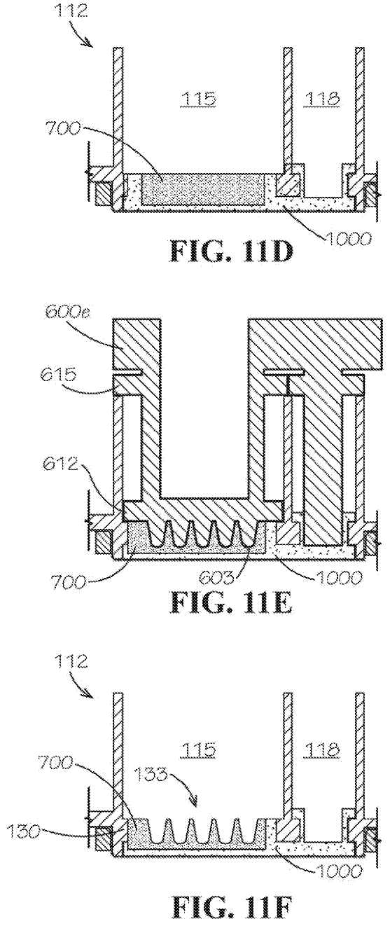

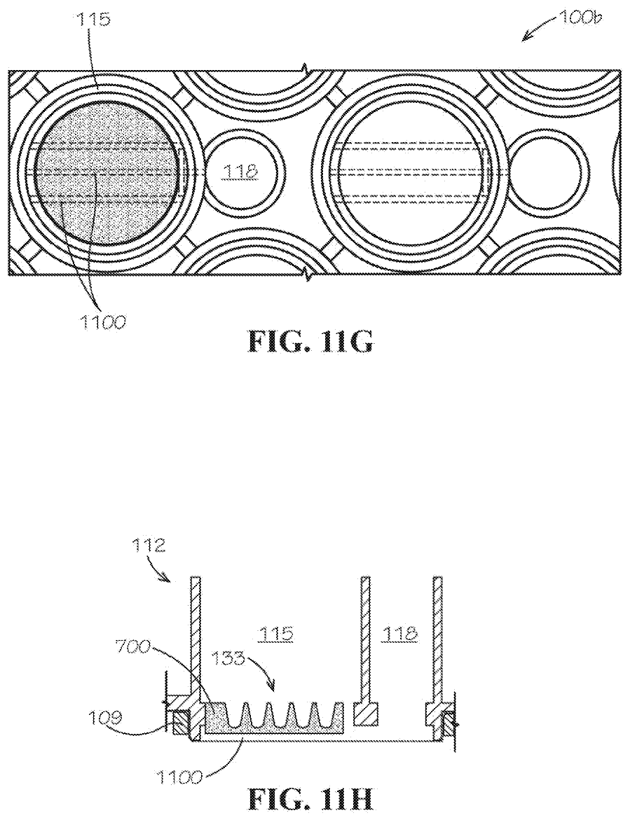

Turning now to FIGS. 10A-10G, shown is an example process for creating channels 1100 (FIGS. 11G and 11H) in a microwell configuration, in accordance to various embodiments of the present disclosure. FIGS. 10A-10G illustrate a cross-sectional view of the well unit 112 of the microplate 100a, in accordance to various embodiments of the present disclosure.

Starting with FIG. 10A, shown is an example of a cross-sectional view of a well unit 112 of the microplate 100 comprising a hydrogel 1000 deposited at a bottom of primary well section 115 and secondary well section 118 for the well unit 112. The hydrogel 1000 is used to form channel configurations in the hydrogel 700 that is deposited into the well units 112 over the hydrogel 1000, and is used to form the microwell structure 133.

The hydrogel 1000 differs from the hydrogel 700 in at least gelling and liquifying properties. In various examples, the hydrogel 1000 comprises a liquid when cooled to temperatures in the range of about 4-10° C. or less. In addition, the hydrogel 1000 forms into a gel at about 10° C. or higher. In contrast, the hydrogel 700 remains a gel at the temperature where the hydrogel 1000 becomes a liquid.

In various examples, the hydrogel 1000 is a poloxamer. In other examples, the hydrogel 1000 comprises Matrigel® (gelatinous protein mixture secreted by Engelbreth-Holm-Swarm mouse sarcoma cells; Corning® Life Sciences), basement matrix (BME), Pluronic®, and/or other type of hydrogel that the properties to form the channels in accordance to the various embodiments of the present disclosure, as can be appreciated. In one aspect, the poloxamer is a nonionic triblock copolymer composed of a central hydrophobic chain of polyoxypropylene (e.g., (poly(propylene oxide)) flanked by two hydrophilic chains of polyoxyethylene (e.g., poly(ethylene oxide)). In one aspect, poloxamer has the formula:

HO(C₂H₄O)_{b}(C₃H₆O)ₐ(C₂H₄O)_{b}OH

wherein a is from 10 to 100, 20 to 80, 25 to 70, or 25 to 70, or from 50 to 70; b is from to 250, 10 to 225, 20 to 200, 50 to 200, 100 to 200, or 150 to 200. In another aspect, the poloxamer has a molecular weight from 2,000 to 15,000 Daltons (Da), 3,000 to 14,000 Da, or 4,000 to 12,000 Da. Poloxamers useful herein are sold under the tradename Pluronic® manufactured by BASF. In preferred examples, the hydrogel 1000 comprises Pluronic F-127®.

FIG. 10B illustrates an example of a cross-sectional view of a mold insert tool 600c inserted into the primary well orifice 121 of the primary well section 115 of a well unit 112, in accordance with various embodiment of the present disclosure. As can be appreciated, the mold insert tool 600c may comprise a mold configuration corresponding to a desired mold of the hydrogel 1000 deposited in the well unit 112. In particular, while the mold insert tool 600a of FIG. 6 comprises mold fingers 606 for creating the microwell structure 133 of FIG. 7, the mold insert tool 600c used to mold the hydrogel 1000 may comprise a different mold configuration. For example, the mold configuration may comprise a plurality of cavities (not shown) disposed along a transverse plane of a distal end of the mold insert member 603c. The plurality of cavities may be used to form channels 1100 (FIG. 11G) within the hydrogel 1000. According to various examples, the channels 1100 may be used to create a fluid connection between the primary well section 115 and the secondary well section 118.

According to various examples, the cross-section of the mold insert member 603 of the mold insert tool 600c matches a cross-section of the primary well section 115. In the example of FIG. 10B, the distal end of the mold insert member 603 is engaged with the hydrogel 1000 situated in the primary well section 115 and supported by the support ledge 130 and bottom layer sheet 106. In addition, the lower stop extension 612 of the mold insert member 603c is engaged with the upper surface of the support ledge 130 and the upper stop extension 615 is engaged with the top surface of the well plate body 103 thereby restricting downward movement of the mold insert member 603 further into the well opening and controlling the amount of the deposited hydrogel 1000 that will be molded.

In some examples, the deposited hydrogel 1000 is in a liquid form and as the hydrogel 1000 transforms to a gel in response to a temperature change, the portions of the hydrogel 1000 engaged with the mold insert member 603 form into a configuration defined by the mold configuration of the mold insert member 603. In other examples, the injected hydrogel 1000 is a gel. In this example, the temperature of the mold insert member 603 can be adjusted to cause the portions of the hydrogel 1000 engaged with the mold insert member 603 to liquify and form into the configuration defined by the mold configuration of the mold insert member 603c.

FIG. 10C illustrates an example cross-section of a well unit 112 of the microplate 100a of FIG. 1 following the removal of the mold insert tool 600c, in accordance to various embodiments of the present disclosure. In particular, FIG. 10C illustrates a cross-section of the molded hydrogel 1000, as can be appreciated.

FIGS. 10D-10G illustrate an example process for depositing a second type of hydrogel 700 into a bottom of a primary well section 115 of a well unit 112 and molding the second type of hydrogel 700 into a microwell structure 133 (FIG. 7) comprising channels 1100 (FIG. 11G) that are formed by the molded first type of hydrogel 1000, in accordance with various embodiments of the present disclosure. As shown in FIG. 10D, hydrogel 700 is deposited into a bottom of a primary well section 115 of the well unit 112 and layered over of the molded hydrogel 1000 from FIG. 10C. The hydrogel 700 can be deposited into the well using any suitable technique. According to various embodiments, as the hydrogel 700 is deposited over the molded hydrogel 700, thereby taking the form of the configuration of the molded hydrogel 700.

In FIG. 10E, shown is an example of a mold insert member 603 of a mold insert tool 600a inserted into the primary well orifice 121 of the primary well section 115 of a well unit 112, in accordance with various examples of the present disclosure. In the example of FIG. 10D, the mold fingers 606 and the hollow extension 609 engage with the hydrogel 700 situated on top of the molded hydrogel 1000 at the bottom of the primary well section 115. The lower stop extension 612 is engaged with the upper surface of the support ledge 130 and the upper stop extension 615 is engaged with the top surface of the well plate body 103 thereby restricting downward movement of the mold insert member 603 further into the well opening and controlling the amount of the deposited hydrogel 700 that will be molded.

In some examples, the deposited hydrogel 700 is a liquid when the mold insert member 603 initially engages with the hydrogel 700. As the hydrogel 700 cools to the gelling temperature of the hydrogel 700, the portions of the hydrogel 700 engaged with the mold insert member 603a, form into a configuration defined by the mold configuration of the mold insert member 603a. In addition, the lower surface of the hydrogel 700 molds to the configuration of the molded hydrogel 1000, as can be appreciated.

FIG. 10F illustrates an example cross-sectional view of a well unit 112 of the microplate 100a of FIG. 1 following the removal of the mold insert tool 600a in accordance with various embodiments of the present disclosure. In particular, FIG. 10F illustrates the formation of the microwell structure 133 for growing cell cultures, as can be appreciated. As shown in FIG. 10F, the microwell structure 133 is formed over the molded hydrogel 700.

According to various embodiments of the present disclosure, the gelling and liquifying temperature properties of the hydrogel 700 and the hydrogel 1000 differ. In particular, according to various embodiments, the hydrogel 1000 turns to a liquid at a given temperature (e.g., about 4° C. or less) while the hydrogel 700 remains a gel. Once the hydrogel 1000 turns to a liquid form, the hydrogel 1000 can be removed from the well unit 112, leaving the hydrogel 700 in the well unit 112. In some examples, the hydrogel 1000 is removed via diffusion, pipetting, and/or other form of removal as can be appreciated. The remaining hydrogel 700 is molded according to the molded configuration of the hydrogel 1000 and the mold insert tool 600a. For example, the lower portions of the hydrogel 700 corresponding to the created channels 1100 may be suspended over the bottom layer sheet 106.

Turning now to FIG. 10G, shown is an example of a cross-sectional view of the well unit 112 comprising the microwell structure 133 with channels 1100 formed on an underside of the microwell structure 133 following the removal of the hydrogel 1000. In particular, the channels 1100 formed on the underside of the microwell structure 133 facilitate the gravitational flow of liquid between the primary well section 115 and the secondary well section 118 in response to tilting of the microplate 100.

Turning now to FIGS. 11A-11H, shown is an example process for creating channels in a microwell configuration in the microplate 100b, in accordance with various embodiments of the present disclosure. FIGS. 11A-11F and 11H illustrate a cross-sectional view of the well unit 112 of the microplate 100b, in accordance with various embodiments of the present disclosure. FIG. 11G illustrates and example top view of the microplate 100b, in accordance with various embodiments of the present disclosure.

Starting with FIG. 11A, shown is an example of a cross-sectional view of a well unit 112 of the microplate 100b comprising a hydrogel 1000 deposited at a bottom of primary well section 115 and secondary well section 118 for the well unit 112. The hydrogel 1000 is used to form channel configurations in the hydrogel 700 that is deposited into the well units 112 over the hydrogel 1000 and is used to form the microwell structure 133.

FIG. 11B illustrates an example of a cross-sectional view of a mold insert tool 600d inserted into a well unit 112 of the microplate 100b, in accordance with various embodiment of the present disclosure. Similar to the mold insert tool 600b of FIG. 9B, the mold insert tool 600d comprises a mold insert member 603d having a first insert extension 903 and a second insert extension 906 corresponding to the primary well section 115 and secondary well section 118, respectively. The cross-section of the first insert extension 903 matches a shape of the primary well section 115 while the cross-section of the second insert extension 906 matches a shape of the secondary well section 118.

The mold insert tool 600d may differ from the mold insert tool 600b with regard to the mold configuration defined at the distal end of the respective first insert extension 903 and the respective second insert extension 906. For example, the mold configuration of the first insert extension 903 and/or the second insert extension 906 may comprise a plurality of cavities (not shown) disposed along a transverse plane of a distal end of the respective first insert extension 903 and/or the respective second insert extension 906. The plurality of cavities may be used to form channels 1100 (FIG. 11G) within the hydrogel 700. According to various examples, the channels 1100 may be used to create a fluid connection between primary well section 115 and the secondary well section 118.

In the example of FIG. 11B, the distal end of the mold insert member 603c is engaged with the hydrogel 1000 situated in the primary well section 115 and the secondary well section 118 and is supported by the support ledge 130 and bottom layer sheet 106. In addition, the lower stop extension 612 of the mold insert member 603c is engaged with the upper surface of the support ledge 130 and the upper stop extension 615 is engaged with the top surface of the well plate body 103 thereby restricting downward movement of the mold insert member 603 further into the well opening and controlling the amount of the deposited hydrogel 1000 that will be molded.

In some examples, the deposited hydrogel 1000 is in a liquid form and as the hydrogel 1000 transforms to a gel in response to a temperature change, the portions of the hydrogel 1000 engaged with the mold insert member 603d form into a configuration defined by the mold configuration of the mold insert member 603d. In other examples, the injected hydrogel 1000 is a gel. In this example, the temperature of the mold insert member 603d can be adjusted to cause the portions of the hydrogel 1000 engaged with the mold insert member 603d to liquify and form into the configuration defined by the mold configuration of the mold insert member 603c.

FIG. 11C illustrates an example cross-section of a well unit 112 of the microplate 100b following the removal of the mold insert tool 600d, in accordance with various embodiments of the present disclosure. In particular, FIG. 11C illustrates a cross section of the molded hydrogel 1000, as can be appreciated.

FIGS. 11D-10H illustrate an example process for depositing a second type of hydrogel 700 into a bottom of a primary well section 115 of a well unit 112 and over the molded hydrogel 1000, and molding the second type of hydrogel 700 into a microwell structure 133 comprising channels 1100 formed by the molded hydrogel 1000, in accordance with various embodiments of the present disclosure. As shown in FIG. 11D, hydrogel 700 is deposited into a bottom of a primary well section 115 of the well unit 112 and layered over of the hydrogel 1000 from FIG. 11C. The hydrogel 700 can be deposited into the well using any suitable technique. According to various embodiments, as the hydrogel 700 is deposited over the molded hydrogel 1000, thereby taking the form of the configuration of the molded hydrogel 1000.

In FIG. 11E, shown is an example of a mold insert member 603 of a mold insert tool 600e inserted into the well unit 112, in accordance with various examples of the present disclosure. In the example of FIG. 11E, the mold fingers 606 and the hollow extension 609 of the first insert extension 903 engage with the hydrogel 700 situated on top of the molded hydrogel 1000 at the bottom of the primary well section 115. The lower stop extension 612 is engaged with the upper surface of the support ledge 130 and the upper stop extension 615 is engaged with the top surface of the well plate body 103 thereby restricting downward movement of the mold insert member 603 further into the well opening and controlling the amount of the deposited hydrogel 700 that will be molded.

In some examples, the deposited hydrogel 700 is a liquid when the mold insert member 603 initially engages with the hydrogel 700. As the hydrogel 700 cools to the gelling temperature of the hydrogel 700, the portions of the hydrogel 700 engaged with the mold insert member 603, form into a configuration defined by the mold configuration of the mold insert member 603. In addition, the lower surface of the hydrogel 700 molds to the configuration of the molded hydrogel 1000, as can be appreciated.

FIG. 11F illustrates an example cross-sectional view of a well unit 112 of the microplate 100b following the removal of the mold insert tool 600e in accordance with various embodiments of the present disclosure. In particular, FIG. 11F illustrates the formation of the microwell structure 133 for growing cell cultures, as can be appreciated. As shown in FIG. 11F, the microwell structure 133 is formed over the molded hydrogel 1000.

According to various embodiments of the present disclosure, the gelling and liquifying temperature properties of the hydrogel 700 and the hydrogel 1000 differ. In particular, according to various embodiments, the hydrogel 1000 turns to a liquid at a given temperature (e.g., about 10° C. or less) while the hydrogel 700 remains a gel. Once the hydrogel 1000 turns to a liquid form, the hydrogel 1000 can be removed from the well unit 112, leaving the hydrogel 700 in the well unit 112. In some examples, the hydrogel 1000 is removed via diffusion, pipetting, and/or other forms of removal as can be appreciated. The remaining hydrogel 700 is molded according to the molded configuration of the hydrogel 1000 and the mold insert tool 600a.

Turning now to FIG. 11G, shown is an example top view of the microplate 100b showing an example of the channels 1100 formed on the underside of the microwell structure 133 upon removal of the hydrogel 1000. FIG. 11H illustrates an example of a cross-sectional view of the well unit 112 comprising the microwell structure 133 with channels 1100 formed on an underside of the microwell structure 133 following the removal of the hydrogel 1000. The cross-section shown in FIG. 11H corresponds to the cross section of one of the channels 1100 illustrated in FIG. 11G. In particular, the channels 1100 formed on the underside of the microwell structure 133 facilitate the gravitational flow of liquid between the primary well section 115 and the secondary well section 118 in response to a tilting of the microplate 100. For examples, the channels 1100 may be used to provide feeding media or other nutrients to the cellular aggregates deposited on the hydrogel 700.

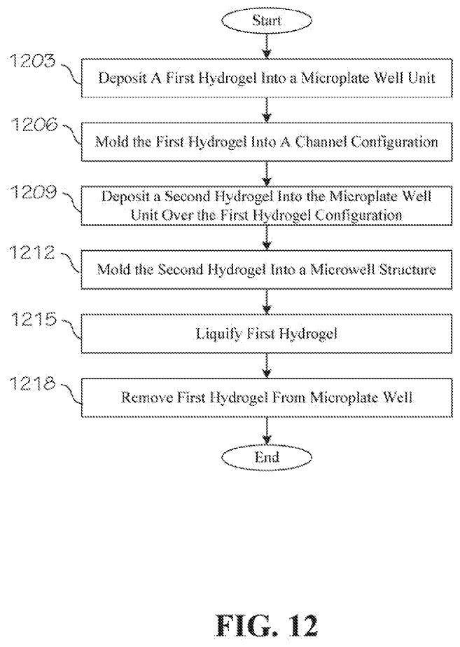

Turning now to FIG. 12, shown is a flowchart of an example method related to the creation of a microwell structure 133 in a well unit 112 of a microplate 100 in accordance to various embodiments of the present disclosure.

Beginning with step 1203, a hydrogel 1000 is deposited into a well unit 112 of a microplate 100. According to various embodiments, the hydrogel 1000 comprises a poloxamer, such as, for example, Pluronic F127®. The hydrogel can be deposited into the well unit by any suitable technique.

At step 1206, the hydrogel 1000 is molded into a channel configuration. For example, a mold insert tool 600 comprising a channel configuration mold may be inserted into one or more well orifices 121, 124 of the well unit 112 until the mold insert tool 600 engages with an appropriate amount of the deposited hydrogel 1000. In some examples, the hydrogel 1000 is in a liquid form and the temperature of the hydrogel 1000 is increased to allow the hydrogel to gel and be molded according to the channel configuration of the mold insert tool 600. In other embodiments, the mold insert tool 600 can be warmed/cooled to the appropriate liquifying temperature of the hydrogel 1000, such that the areas of the hydrogel 1000 engaged with the mold insert tool 600 melt to form the channel configuration defined by the channel configuration mold.

At step 1209, a second hydrogel 700 is deposited into the well unit 112 of the microplate 100 and layered over the hydrogel 1000 that is molded in the channel configuration. According to various embodiments, the second hydrogel 700 comprises agarose and/or other substances suitable for forming the microwell structure 133 of FIG. 7. In some embodiments, the second hydrogel 700 is deposited in a liquid form. In other embodiments, the second hydrogel 700 is deposited in a gel form.

At step 1212, the second hydrogel 700 is molded into a microwell structure 133, in accordance with various embodiments of the present disclosure. For example, a mold insert tool 600 comprising a microwell configuration mold (FIG. 6) can be inserted into the well unit 112 comprising the deposited second hydrogel 700. In particular, the microwell configuration mold can be defined by the mold fingers 606.

Once the mold insert tool 600 is engaged with the second hydrogel 700, the second hydrogel 700 can be molded into a microwell structure 133 as defined by the microwell configuration of the mold insert tool 600. In some examples, the deposited hydrogel 700 is in a liquid form, and as the hydrogel 700 cools to the gelling temperature of the hydrogel 700, the portions of the hydrogel 700 engaged with the mold insert member 603 of the mold insert tool 600, form into a configuration defined by the mold configuration of the mold insert member 603. In other examples, the injected hydrogel 700 is a gel. In this example, the mold insert member 603 can be heated up to a melting temperature of the hydrogel (e.g., greater than about 88° Celsius (C) for agarose) causing the portions of the hydrogel 700 engaged with the mold insert member 603 to melt, thereby forming the microwell structure 133.

At step 1215, a temperature of the microplate 100 can be adjusted to cause the first hydrogel 1000 to liquify thereby, leaving channels 1100 formed in the underside of the microwell structure 133 formed in the second hydrogel 700. For example, the first hydrogel 1000 may liquify at temperatures below 10° C., while the second hydrogel 700 remains a gel. The microplate 100 may be coupled to a temperature control device which may cause the hydrogel 1000 to reach the desired liquifying temperature.

At step 1218, the liquified hydrogel 1000 is removed from well unit 112 of the microplate 100, thereby leaving the hydrogel 700 comprising the microwell structure 133 and channels 1100 formed via the first hydrogel 1000 configuration. The liquified hydrogel 1000 is removed via diffusion, pipetting, and/or other forms of removal as can be appreciated.

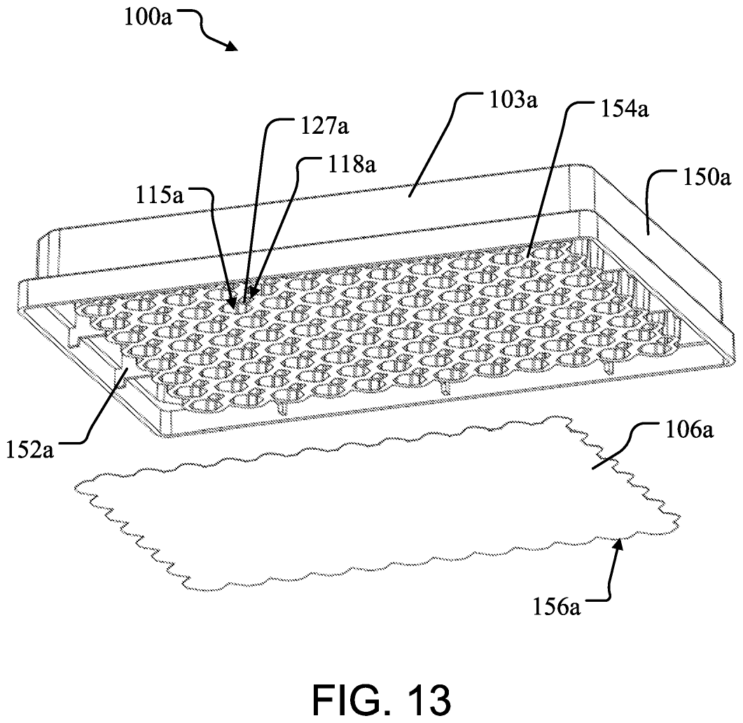

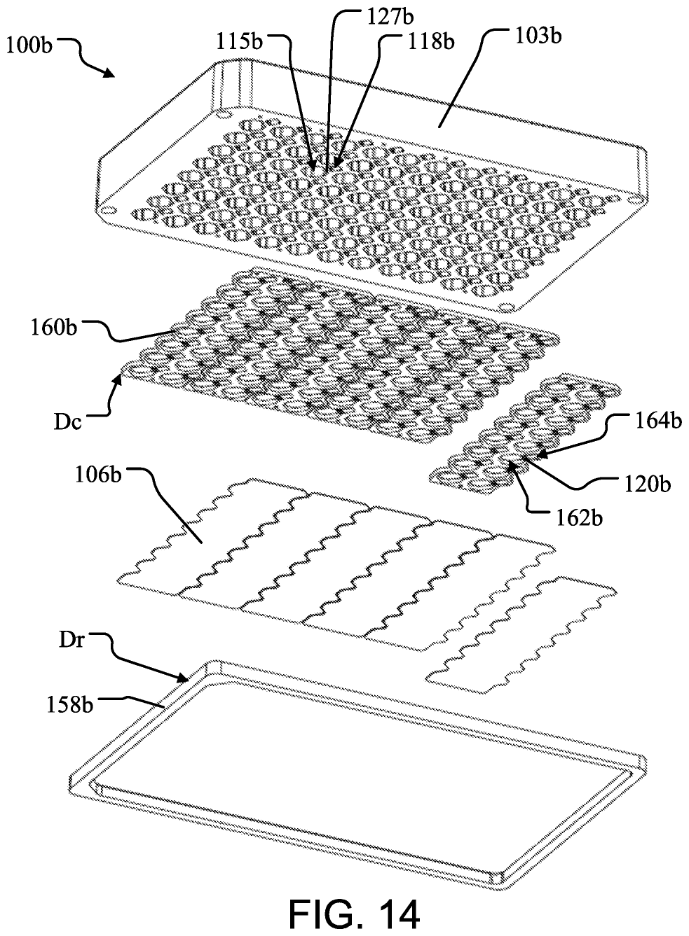

The above examples are depicted in the context of a microplate 100 that includes a clamping frame 109 for securing a bottom sheet 106 to a well plate body 103. Other configurations, for example as depicted in FIGS. 13 and 14 are contemplated that include no clamping frame, or that include other features that improve versatility, manufacturability, performance and/or other characteristics of a microplate. The features of the microplates 100a, 100b, depicted in FIGS. 13 and 14, respectively, are numbered consistent with those of microplate 100 for clarity, but with suffixes “a” and “b”. Not all components are necessarily numbered or described in detail, but the features thereof would be apparent to a person of skill in the art.

FIG. 13 illustrates an example of an exploded perspective view of a microplate 100a according to another embodiment of the present disclosure. The microplate 100a includes an injection molded well plate body 103a. The well plate body 103a is injection molded such that outer walls 150a thereof are substantially hollow; optional struts 152a may be included to increase rigidity of the body 103a. A central well structure 154a forms a plurality of primary well sections 115a and secondary well sections 118a. Here, the primary well sections 115a are larger than the secondary well sections 118a, though other relative sizes are contemplated. One or more channels (not shown) may be formed in shared sidewalls 127a between primary well sections 115a and secondary well sections 118a. For example, the one or more channels may be formed in a bottom-most surface of the central well structure 154a. The depicted microplate 100a also includes a bottom layer sheet 106a. It should be noted that since the bottom-most surface of the central well structure 154a is substantially coplanar across the entire surface thereof (with the exception of any channels formed therein), securing of the bottom layer sheet 106a with a structure such as a clamping frame is impractical. As such, the microplate 100a depicted in FIG. 13 includes a bottom layer sheet 106a that is secured to the body 103a via laser welding, adhesive or solvent bonding, thermocoupling, or other processes, as required for particular component(s) or material(s). In the depicted configuration, the bottom layer sheet 106a includes a contoured edge 156a to prevent the bottom layer sheet 106a from being inadvertently caught or damaged, for example, during stacking of multiple microplates 100a during shipping or storage. In still other examples, the channels between adjacent primary well sections 115a and secondary well sections 118a may be formed on an upper surface of the bottom layer sheet 106a, instead of in the shared sidewalls 127a.

FIG. 14 illustrates an example of an exploded perspective view of a microplate 100b according to another embodiment of the present disclosure. This microplate 100b includes a generally solid well body 103b, which defines the plurality of primary well sections 115b and secondary well sections 118b, divided by shared sidewalls 127b. The body 103b may be formed separate from a lower rim 158b, which has a depth Dr. The microplate 100b also includes a plurality of channel frames 160b. The channel frames 160b may have formed therein one or more channels 120b between openings 162b, 164b that correspond respectively to adjacent primary well sections 115b and secondary well sections 118b. In this example, six channel frames 160b may be utilized with a single well body 103b, but a greater or fewer number may also be used. One advantage to the use of channel frames 160b is that multiple frame configurations (e.g., with different channel 120b configurations) may be utilized simultaneously with a single body 103b. In another example, channel frames 160b having different configurations may be manufactured to be used with a single configuration of a well body 103b, thus reducing the number of custom components that need be manufactured (e.g., a single configuration of a well body 103b may be used in conjunction with multiple configurations of channel frames 160b).

The channel frames 160b may include a depth Dc less than the rim depth Dr. With this lesser depth, a plurality of bottom layer sheets 106b may be secured to one or more of the channel frames 160b so as to close the bottoms of the primary well sections 115b and secondary well sections 118b, while reducing the potential for damage to the bottom layer sheets 106b during stacking or shipping. In other examples, channel frames 160b with more complex configurations of channels 120b may have a depth Dc greater than a standard rim depth Dr. As such, different lower rims 158b having greater depths Dr may be utilized with deeper channel frames 160b.

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±20%, ±15%, ±10%, ±9%, ±8%, ±7%, ±6%, or ±5% of the specified value, e.g., about 1″ refers to the range of 0.8″ to 1.2″, 0.8″ to 1.15″, 0.9″ to 1.1″, 0.91″ to 1.09″, 0.92″ to 1.08″, 0.93″ to 1.07″, 0.94″ to 1.06″, or 0.95″ to 1.05″, unless otherwise indicated or inferred. It is understood that where “about,” “approximate,” or “at or about” is used before a quantitative value, the parameter also includes the specific quantitative value itself, unless specifically stated otherwise.

Any ratios, concentrations, amounts, and other numerical data can be expressed herein in a range format. Such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of less than x′, less than y′, and less than z′. Likewise, the phrase ‘x, y, z, or greater’ should be interpreted to include the specific ranges of ‘about x’, ‘about y’, and ‘about z’ as well as the ranges of ‘greater than x’, greater than y′, and ‘greater than z’. In some aspects, the term “about” can include traditional rounding according to significant figures of the numerical value. In addition, the phrase “about ‘x’ to ‘y’” includes “about ‘x’ to about “y.”

The term “substantially” is meant to permit deviations from the descriptive term that do not negatively impact the intended purpose. All descriptive terms used herein are implicitly understood to be modified by the word “substantially,” even if the descriptive term is not explicitly modified by the word “substantially.”

Although any methods and materials similar or equivalent to those described herein can also be used in the practice or testing of the present disclosure, the various methods and materials suitable for use with the various disclosures disclosed herein are now described. Functions or constructions well-known in the art may not be described in detail for brevity and/or clarity.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A microplate, comprising:
a plate body having an array of wells; and
a gas-permeable sheet secured to a lower portion of the array of wells, the gas-permeable sheet forming a bottom surface of at least a portion of each of the wells.

2. The microplate of claim 1, further comprising a clamping frame comprising an array of collars, individual ones of the collars being positioned around a lower portion of corresponding ones of the wells.

3. The microplate of claim 2, wherein the individual ones of the collars are coupled to corresponding ones of the wells via a friction fit.

4. The microplate of claim 1, wherein the individual wells of the array of wells comprise a primary well section and a secondary well section.

5. The microplate of claim 4, wherein the primary well section and the secondary well section are fluidly connected to one another.

6. The microplate of claim 4, wherein the gas-permeable sheet forms the bottom surface for the primary well section.

7. The microplate of claim 1, wherein individual wells of the array of wells comprise a support ledge protruding from an interior surface of at least one well wall.

8. The microplate of claim 7, wherein the support ledge is ring-shaped.

9. The microplate of claim 7, wherein the support ledge is offset from the bottom surface at a predefined distance.

10. The microplate of claim 7, further comprising hydrogel disposed in the individual wells of the array of wells, the support ledge supporting the hydrogel within the individual wells.

11. The microplate of claim 10, wherein the hydrogel is molded to comprise a plurality of microwells in the individual wells.

12. The microplate of claim 1, wherein the gas-permeable sheet is optically transparent.

13. A microplate, comprising:
a plate body having an array of well units extending from a first end to a second end, individual ones of the well units being formed by at least one well wall and comprising a support ledge that protrudes from an interior surface of the at least one well wall into a well opening, the support ledge being offset from the second end by predefined distance; and
a gas-permeable sheet disposed on an underside of the individual ones of the well units at the second end thereby forming a bottom surface of the individual ones of the well units.

14. The microplate of claim 13, wherein the gas-permeable sheet is optically transparent.

15. The microplate of claim 13, wherein individual well units of the array of well units comprise a primary well section and a secondary well section.

16. The microplate of claim 15, wherein the primary well section and the secondary well section are fluidly connected.

17. The microplate of claim 15, wherein the bottom surface of the primary well section comprises the gas-permeable sheet.

18. The microplate of claim 15, further comprising a clamping frame, the gas-permeable sheet being held against the underside of the individual ones of the well units via the clamping frame.

19. The microplate of claim 18, wherein the clamping frame further comprises an array of collars, where individual ones of the collars are coupled to and positioned around a lower portion of a corresponding one of the well units.

20. The microplate of claim 19, wherein the individual ones of the collars are coupled to the corresponding ones of the well units via a friction fit.

21. The microplate of claim 13, wherein the support ledge is sized and positioned to provide support for an amount of hydrogel injected into the individual ones of the well units.

22. The microplate of claim 13, wherein the support ledge is ring-shaped.

23. The microplate of claim 13, further comprising hydrogel disposed in the individual well units of the array of well units, the support ledge supporting the hydrogel within the individual well units.

24. The microplate of claim 23, wherein the hydrogel is molded to comprise a plurality of microwells in the individual ones of the well units.

25-55. (canceled)
